(19) United States
(12) Patent Application Publication
(10) Pub. No.: US 2011/0105406 A1
(43) Pub. Date: May 5, 2011

(54) Transparent Cosmetic Compositions

(75) Inventors: Chengwu Li, Beijing (CN); Xincheng Hu, Beijing (CN)

(73) Assignee: The Proctor & Gamble Company, Cincinnati, OH (US)

(21) Appl. No.: 12/915,381

(22) Filed: Oct. 29, 2010

(30) Foreign Application Priority Data
Oct. 29, 2009 (CN) CN2009/001203
Sep. 2, 2010 (CN) CN2010/001335

(51) Int. Cl. A61K 8/64; A61K 8/49; A61K 8/60; A61K 8/41; A61K 8/44; A61Q 19/00

(52) U.S. Cl. 514/18.8; 514/358; 514/33; 514/636; 514/23; 514/563; 514/356

(57) ABSTRACT

The present invention relates to a cosmetic composition comprising: a) from about 0.05% to about 10% of a salt form active; b) from about 0.001% to about 2% of a thickening agent; and c) water, wherein the composition has a turbidity no higher than about 10 NTU, and has a pH in the range of about 5.0 to about 6.8.

### FIELD OF THE INVENTION

The present invention relates to transparent cosmetic compositions. Such compositions provide skin whitening, moisturization and/or conditioning as well as attractive transparent appearance.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage.

Currently, there are a number of personal care products that are available to consumers, which are directed toward improving the health and physical appearance of keratinous tissues such as the skin, hair, and nails by delaying, minimizing or even eliminating skin wrinkling and other histological changes typically associated with the aging of skin or environmental damage to human skin. Consumers prefer topically applied products which are not only effective, but also safe and pleasant to use.

While delivery of various skin care actives or compounds that can help to condition the skin are important, it is also important that the product has a pleasant appearance and feel, both prior to and after application to deliver the consumer enjoyable in-use experience. A clear and transparent appearance of cosmetic products has advantages in the market since it can be attributed pureness, mildness, cleanliness, freshness or lightness to consumers. Another benefit of a clear appearance, in combination with a transparent packaging, is that the consumer is readily able to view and inspect the product.

Some of effective skin care actives, usually salt form actives, are known to be difficult to formulate especially in an aqueous environment due to charges in the material which may finally cause significant precipitation or phase separation in the composition which eventually make the final product translucent or turbid.

Based on the foregoing, there is a need to provide cosmetic products incorporating a salt form active without compromising a pleasant transparent appearance of the products.

None of the existing art provides all of the advantages and benefits of the present invention.

### SUMMARY OF THE INVENTION

The present invention relates to a cosmetic composition comprising a) from about 0.05% to about 10% of a salt form active; b) from about 0.001% to about 2% of a thickening agent; and c) water, wherein the composition has a turbidity no higher than about 10 NTU, and has a pH in the range of from about 5.0 to about 6.8.

The present invention also relates to a method of preparing a cosmetic composition comprising: (a) preparing an aqueous phase comprising a thickening agent: and (b) adding a solution comprising a salt form active to the aqueous phase.

These and other features, aspects, and advantages of the present invention will become evident to those skilled in the art from a reading of the present disclosure.

### BRIEF DESCRIPTION OF THE DRAWINGS

FIG. 1 is a photo showing transparency of several embodiments of the invention and a comparative example.

### DETAILED DESCRIPTION OF THE INVENTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The term “salt form actives” as used herein refer to skin care actives which dissociate in an aqueous environment such as an aqueous solution to their respective positively or negatively charged components, or skin care actives which contains an electronic charge in an aqueous environment.

The term a “salt-tolerance thickening agent” as used herein refers to a thickening agent which does not lose a significant portion of its ability to thicken in the presence of salt.

The term “skin care actives,” or “actives,” as used herein refers to compounds that, when applied to the skin, provide a benefit or improvement to the skin. It is to be understood that skin care actives are useful not only for application to skin, but also to hair, nails and other mammalian keratinous tissue.

The compositions of the present invention can include, consist essentially of, or consist of, the components of the present invention as well as other ingredients described herein.

All percentages, parts and ratios are based upon the total weight of the skin care compositions of the present invention, unless otherwise specified. All such weights as they pertain to listed ingredients are based on the active level and, therefore; do not include carriers or by-products that may be included in commercially available materials, unless otherwise specified.

All publications cited herein are hereby incorporated by reference in their entirety.

The compositions of the present invention provide cosmetic products which can provide excellent skin benefits such as high moisturisation, whitening and anti-wrinkle as well as preferable transparent appearance.

The composition of the present invention comprises a thickening agent. Without being bound by theory, thickening agents may build the micro structure in the carrier solution such as netlike structure and cross-linkage structure. Such micro structure may build space hindrance/block in a composition, and help to hold and prevent a salt form active from agglomerating together or crystallization.

The composition of the present invention has a pH in the range of from about 5.0 to about 6.8, preferably in the range of from about 5.5 to about 6.5, more preferably in the range of from about 5.8 to about 6.5.

Transparency of a composition may be negatively affected when a pH of the composition is lower than pH 5.0. There is higher skin irritation risk if pH value is above pH 6.8.

The composition of the present invention is substantially transparent. Transparency of liquid compositions may be characterized by a turbidity measured by a commercially available turbidimeter such as Turbidimeter BTC-464 (Model 2100P, HACH Company, USA), as presented in the TEST METHODS.

The composition of the present invention comprises at least one salt form active, at least one thickening agent and water.

The composition of the present invention preferably contains one or more skin care actives in addition to the salt form active. The nature of the actives and other ingredients depending on their nature can be introduced into the aqueous phase or into one of the oil phases of the present emulsion.

The compositions herein may also include a wide variety of other ingredients. The compositions of the present invention are described in detail hereinafter.

### Salt Form Actives

The compositions according to the present invention comprise a salt form active from 0.01% to 9%, preferably from about 0.05% to 5% and more preferably from 0.1% to 2% by weight of the composition.

Examples of the salt form active in the present invention include, but are not limited to undecenoyl phenylalanine available from Seppic as Sepiwhite MSH, cetylpyridinium chloride, glycyrrhizin, hexamidine, Olivem, and salts thereof.

The salt form actives in the compositions of the present invention may comprises a counter ion, resulted from organic neutralizers. When the counter ion is an cation, a non-metal counter ion, resulted from neutralization with organic neutralizers, such as ammonium ion, trialkanolamine ion, aminomethyl propanol ion, aminomethyl porpandiol ion, and tromethamine ion are preferred. Triethanolamine ion and aminomethyl propanol ion are more preferable.

### Thickening Agents

The composition of the present invention comprises from about 0.001% to about 2%, preferably from about 0.005% to about 1.5%, more preferably from about 0.007% to about 1.0% of thickening agents, including thickeners, gelling agents, and structuring agents. The level and species of the thickening agent are selected according to the compatibility with other components, and other desired characteristic of the product.

Nonlimiting classes of thickening agents include polysaccharides, gums, starch and starch derivatives, carboxylic acid polymers and cationic polymers. Useful herein are salt-tolerance polymers such as polysaccharides, gums, and starch and starch derivatives.

i) Polysaccharides

A wide variety of polysaccharides are useful herein. “Polysaccharides” refer to gelling agents that contain a backbone of repeating sugar (i.e., carbohydrate) units. Non-limiting examples of polysaccharide gelling agents include those selected from the group consisting of cellulose, hydroxyethylcellulose, hydroxyethyl ethylcellulose, hydroxypropylcellulose, hydroxypropyl methylcellulose, methyl hydroxyethylcellulose, microcrystalline cellulose, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the trade name Natrosol® CS Plus from Aqualon Corporation. Another example of preferably polysaccharides polymers is plant-originated polysaccharides such as Tremeall hyaluronic acid which is sold under the tradename WSK from Shanghai Huiwen Biotech.

Other useful polysaccharides include scleroglucans comprising a linear chain of (1-3) linked glucose units with a (1-6) linked glucose every three units, a commercially available example of which is Clearogel™ CS11 from Michel Mercier Products Inc.

ii) Gums

Non-limiting examples of natural gums include guar gums, locust bean gum, xanthan gum and mixtures thereof.

iii) Starch and Starch Derivatives

Non-limiting examples include Structure Solance® a modified potato starch, and Structure XL®, a hydroxypropyl starch phosphate, both commercially available from National Starch.

iv) Carboxylic Acid Polymers

These polymers are compounds containing one or more monomers with an ionizable carboxylic acid group, such as monomers derived from acrylic acid, substituted acrylic acids, and salts of these acrylic acids and the substituted acrylic acids.

Examples of commercially available carboxylic acid polymers useful herein include the carbomers, which are homopolymers of acrylic acid crosslinked with allyl ethers of sucrose or pentaerytritol. Examples of carbomers are the Carbopol® 900 series from Noveon (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e. C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen® TR-1, and Pemulen® TR-2, and Ultrez-21® from Noveon. Also available are sodium acrylate copolymers, such as Luvigel®EM from BASF, Salcare SC-91® from Ciba Specialty Chemicals Corporation, and acrylate/acrylamide copolymers, such as Polymer EX-617 from Noveon.

v) Cationic Polymers

The first aqueous phase of the present invention can optionally comprise cationic polymeric thickening agent, including cationic crosslinked polyacrylate polymers. Examples of useful cationic polymers are polyquaternium-32, available as Salcare®SC-92, and polyquaternium-37 available as Salcare®SC-95 and SC-96, all from Ciba Specialty Chemicals Corporation. Additional cationic polymers are those described in U.S. Pat. No. 5,100,660, U.S. Pat. No. 4,849,484, U.S. Pat. No. 4,835,206, U.S. Pat. No. 4,628,078, U.S. Pat. No. 4,599,379, and EP 228,868

### Water

The cosmetic compositions of the present invention comprise water preferably from 10% to 95, more preferably from about 30% to 90% and more preferably from 50% to 80% by weight of the composition.

### Skin Care Actives

The compositions of the present invention may preferably include at least one skin care active in addition to at least one salt form active. Without being bound by theory, it is believed the present compositions provide versatility in formulating a variety of actives.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Vitamin B₃ Compounds

Vitamin B₃ compound such as niacinamide is a preferred skin care active for use herein. The present invention preferably includes from about 0.1% to about 20%, more preferably from about 0.5% to about 10%, even more preferably from about 2% to about 5% of a vitamin B₃ compound.

As used herein, “vitamin B₃ compound” means a compound having the formula:

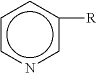

wherein R is —CONH₂ (i.e., niacinamide), —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B₃ compounds, azelaic acid, butyl hydroxy anisole, gallic acid and its derivatives, hydroquinoine, kojic acid, arbutin, mulberry extract, undecylenoyl phenylalanine, and mixtures thereof. Use of combinations of whitening agents is also believed to be advantageous in that they may provide whitening benefit through different mechanisms.

When used, the compositions preferably contain from about 0.01% to about 10%, more preferably from about 0.1% to about 5%, by weight of the composition, of a whitening agent.

Ascorbic acid compounds are useful whitening agents, and include compounds having the formula (I):

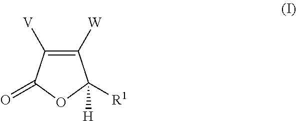

wherein V and W are independently —OH; R¹ is —CH(OH)—CH₂OH; salts thereof; and derivatives thereof.

Preferably, the ascorbic acid compound useful herein is an ascorbic acid salt thereof such as the non-toxic alkali metal, alkaline earth metal and ammonium salts commonly known by those skilled in the art including, but not limited to, the sodium, potassium, lithium, calcium, magnesium, barium, ammonium and protamine salts which are prepared by methods well known in the art; or a derivative thereof such as ascorbyl glucoside.

Flavonoids

The compositions of the present invention may contain a flavonoid compound. Flavonoids are broadly disclosed in U.S. Pat. Nos. 5,686,082 and 5,686,367, both of which are herein incorporated by reference.

Preferred for use herein are substituted flavanones, substituted flavones, substituted chalcones, substituted isoflavones, and mixtures thereof. Some examples of these flavonoids are selected from the group consisting of glucosyl hesperidin, glucosyl rutin, glucosyl myricitrin, glucosyl isoquercitrin, glucosyl quercitrin, methyl hesperidin, and mixtures thereof.

When used, the compositions preferably contain from about 0.01% to about 10%, more preferably from about 0.05% to about 5%, by weight of the composition, of a flavonoid compound.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition.

Sugar Amines

The compositions of the present invention may include a safe and effective amount of a sugar amine, which are also known as amino sugars. As used herein, “sugar amine” refers to an amine derivative of a six-carbon sugar. Preferably, the composition contains from about 0.001% to about 20%, more preferably from about 1% to about 10%, even more preferably from about 2% to about 5%, by weight of the composition, of the sugar amine. Examples of sugar amines that are useful herein include glucosamine, N-acetyl glucosamine, mannosamine. N-acetyl mannosamine, galactosamine. N-acetyl galactosamine. Preferred for use herein is glucosamine. Additionally, combinations of two or more sugar amines may be used.

### Emulsifiers

The composition of the present invention may contain an emulsifier, useful for dispersing or suspending the oil phases, or oily compounds such as perfume within the aqueous phase. The composition may comprise from about 0.001% to about 5%, preferably from about 0.01% to about 3% of at least one emulsifier.

A wide variety of emulsifiers such as nonionic emulsifiers, anionic emulsifiers, cationic emulsifiers and amphoteric emulsifiers can be employed herein, and nonionic emulsifiers are preferable.

In one embodiment, non-limiting examples of which include nonionic emulsifiers such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Nonlimiting examples of other emulsifiers for use herein include: polyethylene glycol 20 sorbitan monolaurate (polysorbate 20), steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, Glycereth-25 PCA Isostearate, and mixtures thereof.

In another embodiment, the emulsifier is a silicone emulsifier, including organically modified organopolysiloxanes (silicone surfactants) such as dimethicone copolyols.

### Humectants

The compositions of the present invention may comprise one or more humectants.

Suitable humectants include, but not limited to, polyhydric alcohols such as polyalkylene glycols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof.

### Emollients

The compositions of the present invention further may comprise emollients.

Suitable emollients include, but are not limited to, hydrocarbons, fatty acids, fatty alcohols and esters.

### Sunscreen Agents

The compositions of the present invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93, and further include terephthalylidene dicamphor sulfonic acid (Mexoryl™ SX).

### Composition Preparation

The composition of the present invention is generally prepared by conventional methods such as are known in the art of making topical compositions. Such methods typically involve mixing of the ingredients in one or more steps to a relatively uniform state, with or without heating, cooling, application of vacuum, and the like.

As an example, water main mix tank containing a thickening agent may be prepared, and added with a separately prepared solution containing a salt form active, followed by preservative premix solution and perfume premix solution.

In one embodiment, the composition of the present invention is prepared by separately preparing an aqueous phase comprising a thickening agent, and a solution comprising a salt form active, and mixing the aqueous phase and the solution. In preparation of a solution comprising a salt form active, the solution may be heated to about 60° C. or above to dissolve actives completely, if needed.

In another embodiment, the composition of the present invention is prepared by separately preparing an aqueous phase comprising a thickening agent, and a solution comprising a salt form active wherein the salt form active is neutralized, and mixing the aqueous phase and the solution. In preparation of a solution comprising a salt form active, the solution may be heated to about 60° C. or above to dissolve actives completely, if needed.

### Product for Use

In preferred embodiments, the composition of the present invention is substantially transparent liquid having turbidity no more than about 10 NTU with or without color. In one preferred embodiment, the composition of the present invention is transparent having turbidity no more than about 7 NTU.

In another preferred embodiment, the composition of the present invention provides an aqueous solution having a viscosity lower than 10,000 cps.

In another preferred embodiment, the composition of the present invention is a hydrogel having a viscosity in the range of from about 10,000 cps to about 100,000 cps.

In another preferred embodiment, the composition of the present invention can be packaged in a transparent container through which consumers can view and inspect the composition.

In some of the embodiments, the composition of the present invention may further comprise at least one skin care active, preferably non salt form active.

### Test Methods

### Turbidity Measurement

Turbidity in a solution can be determined by measuring ratio nephelometric signal (90°) which is ratio of the scattered 90° and transmitted light signal. As an example, turbidity herein can be measured by HACH 2100P Turbidimeter from HACH Company, USA.

The measurement is conducted at a temperature of approximately 25° C. The test range of the turbidity can be set automatic mode. The sample is filled into a (Height×width) 60.0×25 mm (2.36×1 in) Borosilicate glass vial (about 15 mL/0.5 oz) with a screw cap. The sample cell in a fixed place is held for more than 2 hours to let the gas/bubbles out, and wiped. After that, a thin film of silicone oil is applied to the outside of the sample cell, which is wiped with soft cloth to obtain an even film over the entire surface. The sample vial is put into the test hole of the HACH 2100P Turbidimeter, and a turbidity of the sample is measured.

### Viscosity Measurement

A product viscosity is measured by a commercially available viscometer like BROOKFIELD DV II+Viscometer with UL Adapter and UL spindle (BROOKFIELD ENGINEERING LABORATORIES, INC.) at 50 rpm/min at 25° C.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-9 and Comparative Example 1

Examples 1-9 represent non-limiting examples of skin care compositions described herein, suitable for application to keratinous tissue. One comparative example is also described. Compositions are prepared by conventional methods from the following components.

TABLE 1

| | | | | | | | | | | |
|---|---|---|---|---|---|---|---|---|---|---|
| | | | | | | | | | | Com |
| Ingredients | EX 1 | EX 2 | EX 3 | EX 4 | EX 5 | EX 6 | EX 7 | EX 8 | EX 9 | EX 1 |

Mix A

| | | | | | | | | | | |
|---|---|---|---|---|---|---|---|---|---|---|
| Natrosol^{*1} | — | — | 0.01 | 0.05 | — | — | — | — | 1 | — |
| WSK^{*2} | 0.01 | 0.05 | — | — | 0.05 | 0.05 | 0.05 | — | — | — |
| Carbopol 981^{*3} | — | — | — | — | — | — | — | 0.1 | — | — |
| Citric acid | — | — | — | — | — | — | — | — | — | 0.09 |
| Water | q.s. | q.s. | q.s. | q.s. | q.s. | q.s. | q.s. | q.s. | q.s. | q.s. |
| | to | to | to | to | to | to | to | to | to | to |
| | 100 | 100 | 100 | 100 | 100 | 100 | 100 | 100 | 100 | 100 |
| Niacinamide | 5 | 5 | 5 | 5 | 5 | 5 | 5 | 5 | 5 | 5 |

Mix B

| | | | | | | | | | | |
|---|---|---|---|---|---|---|---|---|---|---|
| Sepiwhite | 1 | 1 | 1 | 1 | 0.1 | — | — | 0.1 | 0.1 | 0.1 |
| MSH^{*4} | | | | | | | | | | |
| Elastab ® | — | — | — | — | — | 0.1 | 0.05 | — | — | — |
| HP100^{*5} | | | | | | | | | | |
| Triethanolamine | 0.5 | 0.5 | 0.5 | 0.5 | 0.05 | — | — | 0.05 | 0.05 | 0.05 |
| Pentylene | 4 | 4 | 4 | 4 | 4 | 4 | 4 | 4 | 4 | 4 |
| Glycol | | | | | | | | | | |

Mix C

| | | | | | | | | | | |
|---|---|---|---|---|---|---|---|---|---|---|
| Methyl paraben | 0.15 | 0.15 | 0.15 | 0.15 | 0.15 | 0.15 | 0.15 | 0.15 | 0.15 | 0.15 |
| Ethyl paraben | 0.02 | 0.02 | 0.02 | 0.02 | 0.02 | 0.02 | 0.02 | 0.02 | 0.02 | 0.02 |
| Benzyl Alcohol | 0.2 | 0.2 | 0.2 | 0.2 | 0.2 | 0.2 | 0.2 | 0.2 | 0.2 | 0.2 |
| Butylene Glycol | 6 | 6 | 6 | 6 | 6 | 6 | 6 | 6 | 6 | 6 |
| Dipropylene | 2 | 2 | 2 | 2 | 2 | 2 | 2 | 2 | 2 | 2 |
| Glycol | | | | | | | | | | |

Mix D

| | | | | | | | | | | |
|---|---|---|---|---|---|---|---|---|---|---|
| Perfume | 0.01 | 0.01 | 0.01 | 10.01 | 0.01 | 0.01 | 0.01 | 0.01 | 0.01 | 0.01 |
| Pyroter GPI-25^{*6} | 0.15 | 0.15 | 0.15 | 0.15 | 0.15 | 0.15 | 0.15 | 0.15 | 0.15 | 0.15 |

^{*1}Natrosol (Hydroxyethylcellulose): Available from Hercules Inc.
^{*2}WSK (Tremeall hyaluronic acid): Available from Shanghai Huiwen Biotech
^{*3}Carbopol 981(Carbomer): available from Noveon
^{*4}Sepiwhite MSH (undecylenoyl phenylalanine): Available from Seppic
^{*5}Elastab ® HP100 (hexamidine isethionate): Available from Laboratoires Serobiologiques
^{*6}Pyroter GPI-25 (Glycereth-25 PCA Isostearate): Available from Ajinomoto

The compositions of all the Examples and Comparative Example can be made as follows.

(1) Mix A: All ingredients are mixed in a vessel using a suitable mixer (e.g., Anchor blade, propeller blade, or IKA T25) until the phase becomes homogenous.

(2) Mix B: All ingredients are mixed in a vessel using a suitable mixer (e.g., Anchor blade, propeller blade, or IKA T25) until the phase becomes homogenous. The phase can be heated to about 60° C. or above to dissolve actives completely, if needed.

(3) Mix C: Add other water soluble ingredients and mix until the phase is homogenous. Solid ingredients, if any, can be pre-dissolved in part of water and then blend into the aqueous phase.

(4) Slowly add Mix B into Mix A and mix until batch is homogenous. Slowly add Mix C into the mixture of Mix A and Mix B and mix the obtained mixture until it becomes homogenous. Mix A can be mixed with Mix C prior to mixing Mix A with Mix B.

(5) Add Mix D and mix up to be homogenously.

### Examples 10-13 and Comparative Example 2

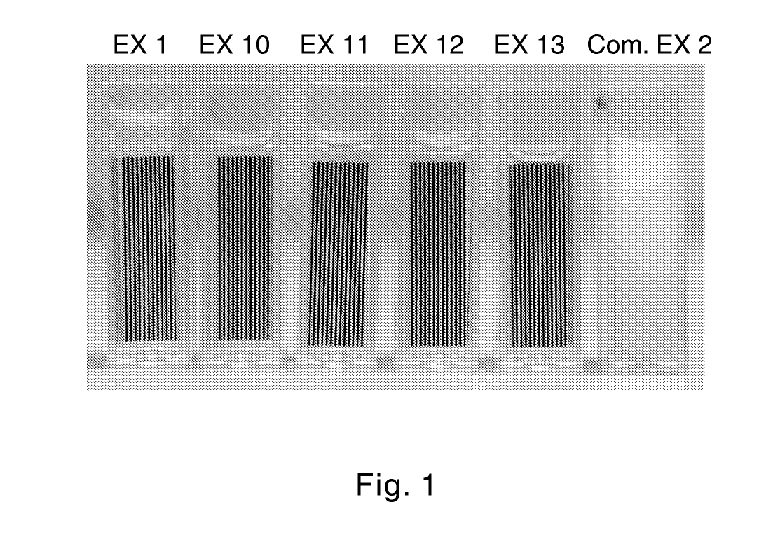

Compositions of Examples 10-13 and Comparative Example 2 were prepared by adding a different amount of 5% citric acid solution into Example 1 composition. Turbidities of Examples 1, 8, 9 and 10-13, and Comparative Examples 1 and 2 were measured according to TURBIDITY MEASUREMENT using HACH 2100P Turbidimeter (HACH Company, USA), and are shown in Table 2. The test range of the turbidity was set to automatic mode. Each composition of Examples 1, 10-13 and Comparative Example 2 was placed in a 4 ml clear cell with a vertical line background. A photo of the cells was taken, and is provided in FIG. 1.

TABLE 2

| | | | | | | | | Com | Com. |
|---|---|---|---|---|---|---|---|---|---|
| | EX 1 | EX 8 | EX 9 | EX 10 | EX 11 | EX 12 | EX 13 | EX 1 | EX 2 |
| pH | 6.808 | 6.560 | 5.160 | 6.039 | 5.775 | 5.722 | 5.696 | 5.690 | 4.800 |
| Turbidity | 0.37 | 3.34 | 2.18 | 1.01 | 2.46 | 3.67 | 5.73 | Above | Above |
| (NTU) | | | | | | | | 1000 | 1000 |

It is understood that the foregoing detailed description of examples and embodiments of the present invention are given merely by way of illustration, and that numerous modifications and variations may become apparent to those skilled in the art without departing from the spirit and scope of the invention: and such apparent modifications and variations are to be included in the scope of the appended claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising:
a) from about 0.05% to about 10% of a salt form active;
b) from about 0.001% to about 2% of a thickening agent; and
c) water,
wherein the composition has a turbidity no higher than about 10 NTU, and has a pH in the range of from about 5.0 to about 6.8.

2. The composition according to claim 1, wherein said salt form active is selected from the group consisting of undecylenoyl phenylalanine, cetylpyridinium chloride, glycyrrhizin, hexamidine, olivem and mixtures thereof.

3. The composition according to claim 1, wherein said composition is a liquid form.

4. The composition according to claim 3, wherein said composition has a viscosity lower than about 10,000 cps.

5. The composition according to claim 4, wherein said composition is an aqueous solution.

6. The composition according to claim 1, wherein said composition has a viscosity in the range of from about 10,000 cps to about 100,000 cps.

7. The composition according to claim 1, wherein said composition has a turbidity no more than about 7 NTU.

8. The composition according to claim 1, wherein said composition has a pH in the range of from about 5.5 to about 6.5.

9. The composition according to claim 1, wherein said salt form active comprises a non-metal counter ion selected from the group consisting of ammonium ion, trialkanolamine ion, aminomethyl propanol ion, aminomethyl porpandiol ion, tromethamine ion, and a combination thereof.

10. The cosmetic composition according to claim 1, wherein said thickening agent is selected from the group consisting of polysaccharides, gums, starch and starch derivatives, carboxylic acid polymers, cationic polymers and a combination thereof.

11. The cosmetic composition according to claim 10, wherein said thickening agent is selected from the group consisting of polysaccharides, gums, starch and starch derivatives, and a combination thereof.

12. The composition according to claim 1, wherein said composition further comprises a skin care active selected from the group consisting of vitamin B₃ compounds, whitening agents, peptides, sugar amines, and mixtures thereof.

13. The composition according to claim 12, wherein said skin care active is vitamin B₃ compounds.

14. The composition according to claim 1, wherein said composition is packaged in a transparent container.

15. A method of preparing a cosmetic composition according to claim 1 comprising:
(a) preparing an aqueous phase comprising a thickening agent; and
(b) adding a solution comprising a salt form active to the aqueous phase.

16. The method according to claim 15, wherein said active form active is neutralized before the solution is added to said aqueous phase.
